(19) United States
(12) Patent Application Publication
(10) Pub. No.: US 2025/0120402 A1
(43) Pub. Date: Apr. 17, 2025

(54) ROOT NODULE GROWTH-PROMOTING BACTERIUM MICROBACTERIUM SP. X-18 AND APPLICATION THEREOF

(71) Applicant: NANJING FORESTRY UNIVERSITY, Nanjing (CN)

(72) Inventors: Jiayao ZHUANG, Nanjing (CN); Chao LIU, Nanjing (CN); Yangyang WANG, Nanjing (CN)

(73) Assignee: NANJING FORESTRY UNIVERSITY, Nanjing (CN)

(21) Appl. No.: 17/202,278

(22) Filed: Mar. 15, 2021

Related U.S. Application Data
(63) Continuation of application No. PCT/CN2020/113411, filed on Sep. 4, 2020.

(30) Foreign Application Priority Data
Sep. 29, 2019 (CN) 2019109381942

(51) Int. Cl. A01N 63/20; A01P 21/00; C12N 1/20; C12R 1/01

(52) U.S. Cl. CPC A01N 63/20; A01P 21/00; C12N 1/205; C12R 2001/01

(57) ABSTRACT

A root nodule growth-promoting Microbacterium sp. X-18 and application thereof are provided, relating to the technical field of microorganisms. The root nodule growth-promoting bacterium is classified as Microbacterium sp. X-18, preserved in China Center for Type Culture Collection with a preservation date of Apr. 8, 2019 and a preservation number of CCTCC No: M 2019236; and the preservation address is Wuhan University, Wuhan, China. The Microbacterium sp. X-18 of the disclosure is applied to the leguminous plant Robinia pseudoacacia, which can promote the effective nitrogen fixation of Robinia pseudoacacia, provide the nitrogen required for the growth of Robinia pseudoacacia, and give full play to the symbiotic nitrogen fixation ability of the root nodule growth-promoting growth promoting bacteria and plants, so as to promote the growth and development of Robinia pseudoacacia plants, and has a promising development prospect.

### REFERENCE TO SEQUENCE LISTING

The substitute sequence listing is submitted as an ASCII formatted text filed via EFS-Web, with a file name of “Substitute Sequence Listing GLP-US-SJDL069.TXT”, a creation date of Feb. 4, 2023, and a size of 1857 bytes. The substitute sequence Listing filed via EFS-Web is a part of the specification and is incorporated in its entirety by reference herein.

### TECHNICAL FIELD

The present disclosure relates to the technical field of microorganisms, and more specifically, to a root nodule growth-promoting bacterium Microbacterium sp. X-18 and application thereof.

### BACKGROUND

External-soil spray seeding is a greening technology in which a mixture of soil growth substrate materials and plant seeds are sprayed on the rocky soil slope evenly and at high pressure. The growth substrate material mixture of this technology is prepared with well-selected soil bacteria, which is mainly a greening technology developed for hard slopes such as rocks and for plants to create a suitable growth environment on hard slopes. As one of the important components of the growth substrate material of this technology, the selected soil bacteria play two roles: firstly, it can respond to changes in soil ecological mechanism and environmental stress. accelerate rock erosion, and effectively improve the fusion of the rock wall and the spray seeding substrate interface; secondly, it is to promote the growth and development of plants to ensure the supply of nutrients such as carbon and nitrogen in the stress environment. However, in actual engineering, the use of this technology is subject to many restrictions. On the one hand, the spray seeding substrate is difficult to maintain on the rock surface for a long time. On the other hand, the discovery of soil microorganisms that can promote growth of spraying tree species is rare.

During their life activities, soil microorganisms convert inert nitrogen in the air into ionic nitrogen that can be directly absorbed by vegetation to ensure the nitrogen nutrition of vegetation. At the same time, the stomatal conductance of vegetation treated by microorganisms with a growth-promoting effect can be increased, which can accelerate the gas exchange of plant cells, and increase the intercellular CO; concentration, so that the CO, required for photosynthesis is sufficient, which further improves the photosynthetic rate of plants and promotes the photosynthesis. In addition, microorganisms can decompose incompatible minerals in the soil and degrade inorganic and organic pollutants during their life activities. These all ensure the supply of nutrients required by vegetation in the microenvironment and the normal progress of life activities of vegetation. Therefore, the obvious and targeted selection of growth-promoting bacteria for specific vegetation is one of the keys to the wide application of the external-soil spray seeding greening technology. At present, there are few reports on the selection of suitable legume vegetation nodule-promoting strains for different vegetations in China.

### SUMMARY

In view of the problems existing in the prior art, an objective of the disclosure is to provide a root nodule growth-promoting bacterium Microbacterium sp. X-18, which provides strain support for the establishment of slope plant growth promoting bacteria bank. Another object of the disclosure is to provide an application of the Microbacterium sp. X-18 in promoting the nodulation and nitrogen fixation of Robinia pseudoacacia. It can provide high level of nitrogen and promote the nodulation and nitrogen fixation of Robinia pseudoacacia. Another object of the disclosure is to provide an application of the Microbacterium sp. X-18 in promoting the growth of Robinia pseudoacacia. It can effectively promote the growth of ground diameter, seedling height and leaf area.

Technical solutions of the present disclosure are specifically described as follows.

In a first aspect, the disclosure provides a root nodule growth-promoting bacterium, classified as Microbacterium sp. X-18, preserved in China Center for Type Culture Collection with a preservation date of Apr. 8, 2019 and a preservation number of CCTCC No: M 2019236, and the preservation address is Wuhan University, Wuhan, China.

In a second aspect, the disclosure provides an application of the Microbacterium sp. X-18 in promoting nodulation and nitrogen fixation of Robinia pseudoacacia.

A fermentation broth of the Microbacterium sp. X-18 is diluted and directly watered on the rhizosphere soil of Robinia pseudoacacia seedlings.

The application of claim includes the following steps:
A. preparing strains of Microbacterium sp. X-18 from a slope, and activating the prepared strains on a nutrient agar solid medium at 35° C. for 24 hours;
B. picking up a loop of bacterial paste of the activated Microbacterium sp. X-18 strains with an inoculation loop, adding the bacterial paste to a Luria-Bertani (LB) liquid medium, and shaking the medium under a constant temperature of 35° C. with a frequency of 200 r/min for 24 hours to prepare a seed solution;
C. taking the seed solution with 3% of the inoculum amount, inoculating the taken seed solution into a liquid medium, and culturing with shaking under a temperature of 35° C. with a frequency of 200 r/min for 36 hours to obtain the fermentation broth;
D. diluting the fermentation broth obtained in step C with sterile water and then adding the diluted fermentation broth to potted Robinia pseudoacacia seedlings in a mount of 60 ml/pot.

Further, the liquid medium in step C is 10 g peptone, 3 g yeast powder, 5 g sodium chloride, and 1000 ml sterile water, with a pH of 5.6.

In a third aspect, the disclosure provides an application of the Microbacterium sp. X-18 in promoting the growth of Robinia pseudoacacia.

The application includes the following steps:
A. preparing strains of Microbacterium sp. X-18 from a slope, and activating the prepared strains on a nutrient agar solid medium at 35° C. for 24 hours;
B. picking up a loop of bacterial paste of the activated Microbacterium sp. X-18 strains with an inoculation loop, adding the bacterial paste to a Luria-Bertani (LB) liquid medium, and shaking the medium under a constant temperature of 35° C. with a frequency of 200 r/min for 24 hours to prepare a seed solution;
C. taking the seed solution with 3% of the inoculum amount, inoculating the taken seed solution into a liquid medium, and culturing with shaking under a temperature of 35° C. with a frequency of 200 r/min for 36 hours to obtain the fermentation broth;
D. diluting the fermentation broth obtained in step C with sterile water and then adding the diluted fermentation broth to potted Robinia pseudoacacia seedlings in a mount of 60 ml/pot.

Further, the liquid medium in step C is 10 g peptone, 3 g yeast powder, 5 g sodium chloride, and 1000 ml. sterile water, with a pH of 5.6.

Compared with the prior art, the disclosure has the following beneficial effects. The root nodule growth-promoting Microbacterium sp. X-18 screened out by the disclosure can promote the effective nitrogen fixation of Robinia pseudoacacia, provide the nitrogen required for the growth of Robinia pseudoacacia, give full play to the symbiotic nitrogen fixation ability of root nodule growth-promoting bacteria and plants, and has a promising development prospect.

### BRIEF DESCRIPTION OF THE DRAWINGS

FIG. 1 is a diagram of the colony of Microbacterium sp. X-18 on a nutrient agar solid medium.

### DETAILED DESCRIPTION OF EMBODIMENTS

Hereinafter, the present disclosure will be further described with reference to specific embodiments, but the embodiments are not intended to limit the present disclosure. The methods used in the following embodiments are conventional methods unless otherwise specified. The materials and reagents used in the following embodiments, unless otherwise specified, are commercially available.

### Embodiment 1 Isolation and Identification of Strain

Soil samples were collected from the 5 cm rhizosphere soil on the slopes on both sides of Yueyang Avenue in Yueyang City. The dilution coating plate method is adopted. In a 35° C. incubator, it was cultured on a nutrient agar solid medium (NA medium: peptone 10 g; beef powder 3 g; sodium chloride 5 g; agar 15 g; sterile water 1000 ml) for 2 to 3 days. Different colonies were picked out by naked eye observation, and several different species of single colonies were obtained through repeated streaking and purification.

A single colony was selected and made into a plate, and was sent to Shanghai Jinyu Medical Laboratory for sequencing, and the 16S rDNA gene sequence was obtained, as shown in SEQ ID NO.1. The detected 16S rDNA gene sequence was BLAST aligned with the sequence in the GenBank database. The results showed that the similarity between the strain and Microbacterium chocolatum was 99.75%. The morphological characteristics and 16S rDNA gene sequence were combined and analyzed, and it was identified as Microbacterium sp. X-18.

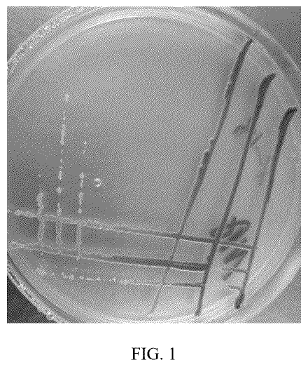

The main biological characteristics of the Microbacterium sp. X-18 were as follows: cultured in the nutrient agar medium (NA solid medium) at 35° C. for 24 hours, as shown in FIG. 1. Colony characteristics: brown, moist, smooth, round, Gram staining for blue purple positive, short rod shape; glucose fermentation experiment:—, no bubbles; lactose fermentation experiment:—, no bubbles; Starch Hydrolysis Experiment:—; indole experiment:—; methyl red experiment:—; V.P. experiment:—; citrate experiment:—; hydrogen sulfide experiment:—.

### Embodiment 2 Ability of the Strain to Promote Nodulation and Nitrogen Fixation of Robinia pseudoacacia

### 1. Preparation of Fermentation Broth:

1) Strains of Microbacterium sp. X-18 were taken from the slope, and the taken strains were activated on a nutrient agar solid medium at 35° C. for 24 hours;
2) A loop of bacterial paste of the activated Microbacterium sp. X-18 was picked up with an inoculation loop and added to the LB liquid medium, and the medium was shaken at a constant temperature of 35° C., at a frequency of 200 r/min, for 24 hours to prepare a seed solution;
3) The seed solution was taken with 3% of the inoculum amount and was inoculated into liquid medium (10 g peptone, 3 g yeast powder, 5 g sodium chloride, and 1000 mL sterile water, pH 5.6). The seed solution was cultured with shaking at a temperature of 35° C., at a frequency of 200 r/min, until the OD₅₆₀ was 0.8-1.2 to obtain the fermentation broth;
4) Before use, the fermentation broth obtained in step 3) was diluted with sterile water by 100 times, and directly sprayed in potted plants of planted Robinia pseudoacacia seedlings.

### 2. Experiment on the Potted Plants

According to 60 ml/pot, the diluted fermentation broth was added to the surrounding of the planted Robinia pseudoacacia seedlings (the actual planting period was 4 months; the first month was the thinning seedling stage, without bacteria application, after the inter seedling stage, the bacteria application began to be calculated). Sterile water was set as blank control group (CK), and the nodulation and nitrogen fixation capacity of Robinia pseudoacacia was analyzed after one quarter observation.

Potted plants were observed and counted. At the 8th week, the plants were carefully dug out, the soil at the roots of the Robinia pseudoacacia was simply cleaned up, and the number of nodules was recorded, and then the plants were replanted into the pots. At the 16th week, the nodules were counted for the last time, and the nodule number and nodule weight were counted and recorded. The results are shown in Table 1.

TABLE 1
Results of nodule number, nodule weight and root dry weight

| | Nodule Number | | | | |
|---|---|---|---|---|---|
| | (Pcs/plant) | | | | |
| | 8th | 12th | 16th | Nodule Weight | Root Dry Weight |
| | week | week | week | (g/Pcs) | (g) |
| X-18 | 0 | 2 | 5 | 0.0083 | 0.51 |
| CK | 0 | 0 | 0 | — | 0.23 |

It can be seen from Table 1 that compared with the blank control group, the nodule number and nodule weight of Robinia pseudoacacia increased significantly after adding Microbacteria sp. X-18, and the root dry weight of Robinia pseudoacacia also increased significantly, with an average increase of 121.74% compared with the blank control group. It can be seen that the strain can promote the nodulation and nitrogen fixation of Robinia pseudoacacia and accelerate the growth and development of its roots. It is a very promising model for the configuration of promoting strains for Robinia pseudoacacia.

### 3. Promoting Experiment

According to 60 ml/pot, the diluted fermentation broth was added to the surrounding of the planted Robinia pseudoacacia seedlings. One month later, the seedlings of Robinia pseudoacacia were thinned to ensure the uniformity of the number of seedlings in each pot. After the first thinning, the ground diameter of the Robinia pseudoacacia seedlings was measured every 30 days using a vernier caliper. The seedling height of the seedlings was measured using a tape measure. On the termination day, a total of 10 upper, middle and lower leaves were selected from each pot, and the leaf areas were measured with a root scanner. The results are shown in Table 2.

TABLE 2
Growth index of Robinia pseudoacaciai seedlings in one quarter

| Processing | Ground diameter | Seedling height | |
|---|---|---|---|
| method | (mm) | (cm) | Leaf area (cm²) |
| X-18 | 5.81 | 51.2 | 51.31 |
| CK | 4.75 | 40.04 | 43.53 |

Note:
P < 0.05.

The ground diameter is used to indicate the size of the trees, and the growing plants generally have a larger ground diameter. It can be seen from Table 2 that the ground diameter of the treated group was 22.32% higher than that of the blank control group.

Seedling height is one of the most basic indicators of plant morphology, which can directly reflect the growth status of vegetation. In general, the seedling height of plants that grow well is relatively high. It can be seen from Table 2 that the seedling height of the treated group was 27.87% higher than that of the blank control group. Leaf area is one of the indicators most closely related to yield. The increase in plant yield can be directly reflected by leaf area. In general, a proper size of leaf area can make full use of light conditions without affecting photosynthesis. It can be seen from Table 2 that the leaf area of the treated group was 17.87% higher than that of the blank control group.

We claim:

1. A root nodule growth-promoting bacterium, classified as Microbacterium sp. X-18, preserved in China Center for Type Culture Collection with a preservation date of Apr. 8, 2019 and a preservation number of CCTCC No: M 2019236; wherein the preservation address is Wuhan University, Wuhan, China.

2. An application of the Microbacterium sp. X-18 of claim 1 in promoting nodulation and nitrogen fixation of Robinia pseudoacacia.

3. The application of claim 2, wherein a fermentation broth of the Microbacterium sp. X-18 is diluted and directly watered on rhizosphere soil of Robinia pseudoacacia seedlings.

4. The application of claim 3, comprising the following steps:
A. preparing strains of Microbacterium sp. X-18 from a slope, and activating the prepared strains on a nutrient agar solid medium at 35° C. for 24 hours;
B. picking up a loop of bacterial paste of the activated Microbacterium sp. X-18 strains with an inoculation loop, adding the bacterial paste to a Luria-Bertani (LB) liquid medium, and shaking the medium under a constant temperature of 35° C. with a frequency of 200 r/min for 24 hours to prepare a seed solution;
C. taking the seed solution with 3% of the inoculum amount, inoculating the taken seed solution into a liquid medium, and culturing with shaking under a temperature of 35° C. with a frequency of 200 r/min for 36 hours to obtain the fermentation broth;
D. diluting the fermentation broth obtained in step C with sterile water and then adding the diluted fermentation broth to potted Robinia pseudoacacia seedlings in a mount of 60 ml/pot.

5. The application of claim 4, wherein the liquid medium in step C is 10 g peptone, 3 g yeast powder, 5 g sodium chloride, and 1000 ml sterile water, with a pH of 5.6.

6. An application of the Microbacterium sp. X-18 of claim 1 in promoting the growth of Robinia pseudoacacia.

7. The application of the Microbacterium sp. X-18 of claim 6, wherein a fermentation broth of Microbacterium sp. X-18 is diluted and directly watered on rhizosphere soil of Robinia pseudoacacia seedlings.

8. The application of claim 7, comprising the following steps:
A. preparing strains of Microbacterium sp. X-18 from a slope, and activating the prepared strains on a nutrient agar solid medium at 35° C. for 24 hours;
B. picking up a loop of bacterial paste of the activated Microbacterium sp. X-18 strains with an inoculation loop, adding the bacterial paste to a Luria-Bertani (LB) liquid medium, and shaking the medium under a constant temperature of 35° C. with a frequency of 200 r/min for 24 hours to prepare a seed solution;
C. taking the seed solution with 3% of the inoculum amount, inoculating the taken seed solution into a liquid medium, and culturing with shaking under a temperature of 35° C. with a frequency of 200 r/min for 36 hours to obtain the fermentation broth;
D. diluting the fermentation broth obtained in step C with sterile water and then adding the diluted fermentation broth to potted Robinia pseudoacacia seedlings in a mount of 60 ml/pot.

9. The application of claim 7, wherein the liquid medium in step C is 10 g peptone, 3 g yeast powder, 5 g sodium chloride, and 1000 mL sterile water, with a pH of 5.6.


SEQUENCE LISTING (1 sequence)

SEQ ID NO: 1 - DNA, 1040 bp, Artificial sequence
  Microbacterium sp.
tagcaatggcggcgtgcttaccatgcagtcgaacggtgaaagagagcttgctctctggatcagtggcgaacgggtgagtaacacgtgagcaacctgccccggactctgggataacagctggaaacagctgctaataccggatacgagctgcgaaggcatcttcggcagctggaaagaatttcggtccgggatgggctcgcggcctatcagctagttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaaacccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttggaactagttgtggggtccattccacggattccgtgacgcagctaacgcattaagttccccgcctggggagtacggccgcaggctaaactcaaggaattgacggggacccgcacagcggcggagcatgcggattaattcgatgcaacgcgaagaccttaccaaggcttgacatatacgagaccgggccagaatgtcaactctttggacacctcgtaacaggtgtgcatgttgtcgtcagctcgtgtcgtgagaatgttggttaagttcccgcaacga